(19) United States
(12) Patent Application Publication
(10) Pub. No.: US 2025/0230136 A1
(43) Pub. Date: Jul. 17, 2025

(54) METHOD FOR PRODUCING KETONES

(71) Applicant: Bayer Aktiengesellschaft, Leverkusen (DE)

(72) Inventor: Karsten VON DEM BRUCH, Leverkusen (DE)

(21) Appl. No.: 18/849,086

(22) PCT Filed: Mar. 16, 2023
(86) PCT No.: PCT/EP2023/056732

§ 371 Date: Sep. 20, 2024

(30) Foreign Application Priority Data
Mar. 21, 2022 (EP) 22163240.9

(51) Int. Cl. C07D 317/72; B01J 23/30

(52) U.S. Cl. CPC C07D 317/72; B01J 23/30

(57) ABSTRACT

The present invention relates to a novel method for preparing compounds of the formula (II) by reacting compounds of the formula (I) with hydrogen peroxide in the presence of a solvent, in the presence of a catalyst, in the presence of a buffer and optionally in the presence of a base.

The present invention relates to a novel method for preparing ketones by oxidation of alcohols with hydrogen peroxide.

Ketones are used inter alia as starting materials for preparing hydantoins. Said hydantoins are in turn important intermediates in the production of pesticides (for example WO 2006/089633).

The preparation of ketones by oxidation of alcohols can be accomplished by various methods that have long been known. In Chem. Com. 2003, 16, 1977, JACS, 1997, 119, 12386 and Bull. Chem. Soc. Japan, 72(10), 1999, 2287-2306, ketones are prepared by reacting alcohols with hydrogen peroxide as oxidant, sodium tungstate dihydrate as catalyst and optionally a hydrogen sulfate-based phase-transfer catalyst. However, this known oxidation of alcohols has considerable shortcomings and disadvantages, in particular the low pH is problematic, which means that acid-labile starting compounds are not suitable for this transformation.

It has now been found that the reaction for the preparation of ketones can advantageously be carried out in such a way that the reaction of alcohols with hydrogen peroxide takes place in the presence of a solvent, in the presence of a catalyst and in the presence of a buffer. A stable pH can if necessary be achieved by metering in a base in parallel to the metered addition of the hydrogen peroxide. This allows even acid-labile starting compounds to react outstandingly well in good yield. It has also been found to be possible to prepare ketones in higher purity. The formation of secondary components is substantially minimized and the ketones can be used directly in further reactions, for example for the preparation of hydantoins, without further purification.

The method of the invention for preparing compounds of the formula (II) can be illustrated by the following scheme:

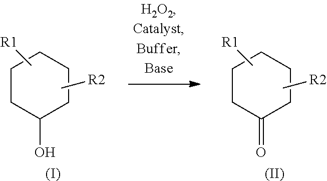

In the formulas (I) and (II)
R¹ and R² and the carbon atom to which they are attached represent a five- or six-membered ketal that is optionally singly or doubly substituted by methyl or ethyl;
R¹ and R² and the carbon atom to which they are attached preferably represent a five-membered ketal.

Particular preference is given to the following compound of the formula (I-1): 1,4-dioxaspiro[4.5]decan-8-ol

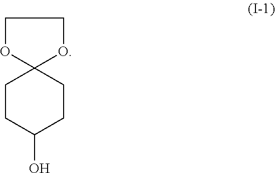 [SMILES: OC1CCC2(CC1)OCCO2]

Particular preference is given to the following compound of the formula (II-1): 1,4-dioxaspiro[4.5]decan-8-one

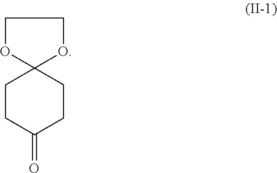 [SMILES: O=C1CCC2(CC1)OCCO2]

Surprisingly, it is possible according to the method of the invention to prepare compounds of the formula (II) in higher yield and in higher purity than is possible according to previous known methods. Moreover, acid-labile starting compounds are also suitable for this reaction.

The compounds of the formula (I) are either commercially available or can be prepared by known methods (for example WO 2006/089633).

### Scheme 1

The course of the method according to the invention is exemplified by the following reaction scheme:

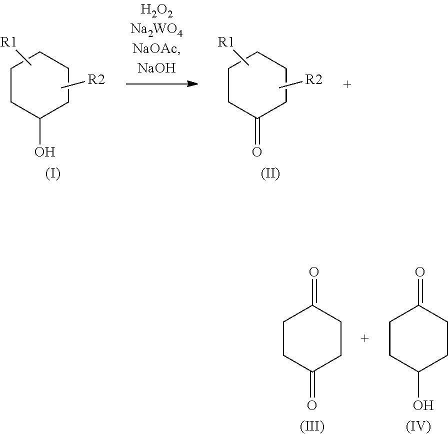

The compounds of the formulas (III) and (IV) are secondary components of the reaction. The formation of secondary components of the formulas (III) and (IV) is substantially minimized by the method according to the invention. This minimization represents a major advantage of the method according to the invention over the method known from the prior art.

The oxidant used is hydrogen peroxide. The hydrogen peroxide used is commercial hydrogen peroxide with a concentration of 10 to 70% hydrogen peroxide in water. Preferably is given to hydrogen peroxide with a concentration of 50% hydrogen peroxide in water.

Examples of solvents used for the reaction according to the method of the invention are water, ketones such as acetone, methyl ethyl ketone, methyl isobutyl ketone or cyclohexanone; tert-butanol; and ethers such as diisopropyl ether, dioxane, tetrahydrofuran or 1,2-dimethoxyethane. Preference is given to using water as solvent.

Examples of suitable catalysts are sodium tungstate, sodium tungstate dihydrate, sodium molybdate or sodium molybdate dihydrate. Preference is given to using sodium tungstate dihydrate. Preference is likewise given to using sodium tungstate.

Examples of suitable buffers are acetates, formates, propionates, phosphates, carbonates or alkoxides of alkali metals or alkaline earth metals or the corresponding ammonium compounds. Preference is given to sodium acetate, potassium acetate, lithium acetate, ammonium acetate, sodium formate, potassium formate, sodium propionate, potassium propionate, sodium butyrate, potassium butyrate, sodium isobutyrate or potassium isobutyrate. Particular preference is given to using sodium acetate, ammonium acetate, lithium acetate, potassium acetate, sodium propionate, sodium butyrate or sodium isobutyrate. Also suitable are buffer mixtures of for example sodium acetate and acetic acid.

Control of the pH during the reaction is important. The pH can be controlled using a base, for example aqueous ammonia, sodium hydroxide, potassium hydroxide or lithium hydroxide. Preference is given to using sodium hydroxide for pH control.

The pH is determined using a calibrated pH electrode.

The amount of oxidant used is 1 to 5 mol per mole of compound of the formula (I), preferably 2 to 4 mol per mole of compound of the formula (I) is used. Particular preference is given to using 2.9 to 3.5 mol per mole of compound of the formula (I).

The oxidant is added over a certain period, preferably the addition takes place within a period of 3 to 5 hours. The oxidant can be added in an even manner over this period.

The catalyst is used in an amount of 0.01 to 0.2 mol per mole of compound of the formula (I), preferably 0.01 to 0.1 mol per mole of compound of the formula (I), more preferably 0.01 to 0.04 mol per mole of compound of the formula (I).

The buffer is used in an amount of 0.01 to 1 mol per mole of compound of the formula (I), preferably 0.01 to 0.5 mol per mole of compound of the formula (I), more preferably 0.05 mol per mole of compound of the formula (I).

Through metered addition in parallel to the hydrogen peroxide, the base ensures a stable pH range. A favourable pH is regarded as being a pH of between 5 and 8. Preference is given to a pH of between 5.5 and 7.5. Particularly preferably, the pH of the reaction mixture is on reaching pH 5.5 always raised to pH 6.5 by adding base. The amount of base for pH control is chosen so as to adjust the pH accordingly.

The reaction can be carried out at temperatures of between 5° and 100° C. Preference is given to temperatures of between 75 and 85° C. Very particular preference is given to a temperature of 80° C.

In a further embodiment, the invention relates to a method for preparing compounds of the formula (II) by reacting compounds of the formula (I) with hydrogen peroxide in the presence of a solvent, in the presence of a catalyst, in the presence of a buffer and in the presence of a base.

In a further embodiment, the invention relates to a method for preparing compounds of the formula (II) by reacting compounds of the formula (I) with hydrogen peroxide in the presence of a solvent, in the presence of a catalyst, in the presence of a buffer and optionally in the presence of a base, wherein the pH is between 5.5 and 7.5.

In a further embodiment, the invention relates to a method for preparing compounds of the formula (II) by reacting compounds of the formula (I) with hydrogen peroxide in the presence of a solvent, in the presence of a catalyst and in the presence of a buffer, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate or sodium tungstate dihydrate as catalyst, in the presence of a buffer and optionally in the presence of a base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of a buffer and optionally in the presence of a base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of sodium acetate, ammonium acetate, lithium acetate, potassium acetate, sodium propionate, sodium butyrate or sodium isobutyrate as buffer and in the presence of sodium hydroxide as base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of sodium acetate as buffer and in the presence of aqueous ammonia, sodium hydroxide, potassium hydroxide or lithium hydroxide as base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of sodium acetate as buffer and in the presence of sodium hydroxide as base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of ammonium acetate as buffer and in the presence of sodium hydroxide as base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of ammonium acetate as buffer and in the presence of aqueous ammonia as base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of lithium acetate as buffer and in the presence of sodium hydroxide as base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of lithium acetate as buffer and in the presence of lithium hydroxide as base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of potassium acetate as buffer and in the presence of sodium hydroxide as base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of potassium acetate as buffer and in the presence of potassium hydroxide as base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of sodium propionate as buffer and in the presence of sodium hydroxide as base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of sodium butyrate as buffer and in the presence of sodium hydroxide as base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of sodium isobutyrate as buffer and in the presence of sodium hydroxide as base, wherein the pH is between 5.5 and 7.5.

In a further embodiment, the invention relates to a method for preparing compounds of the formula (II) by reacting compounds of the formula (I) in water with hydrogen peroxide, in the presence of sodium tungstate dihydrate as catalyst, in the presence of sodium acetate as buffer and in the presence of sodium hydroxide as base, wherein 0.02 to 0.04 mol of sodium tungstate dihydrate per mol of compound of the formula (I) and 3.3 mol of hydrogen peroxide per mol of compound of the formula (I) are used and the pH is between 5.5 and 6.5.

In a further embodiment, the invention relates to a method for preparing compounds of the formula (II) by reacting compounds of the formula (I) in water with hydrogen peroxide, in the presence of sodium tungstate or sodium tungstate dihydrate as catalyst, in the presence of sodium acetate, ammonium acetate, lithium acetate, potassium acetate, sodium propionate, sodium butyrate or sodium isobutyrate as buffer or in the presence of sodium acetate and acetic acid as buffer mixture and optionally in the presence of sodium hydroxide, potassium hydroxide, lithium hydroxide or aqueous ammonia as base, wherein the pH is between 5.5 and 7.5.

The preparation of the compounds of compounds of the formula (II) by the method according to the invention shall be illustrated by the preparation examples that follow:

### EXAMPLES

### Example 1: Preparation of 1,4-dioxaspiro[4.5]decan-8-one

A mixture of 53.7 g (93%, 0.32 mol) of 1,4-dioxaspiro[4.5]decan-8-ol, 75 g of water, 1.3 g (0.016 mol) of sodium acetate and 2.0 g (0.006 mol) of sodium tungstate dihydrate is heated to 80° C. At this temperature, 65.7 g (0.97 mol) of 50% hydrogen peroxide is metered in over a period of 5 h. During this time, the pH of the reaction mixture is on reaching pH 5.5 always raised to pH 6.5 by adding 50% sodium hydroxide solution. In addition, a further 1.0 g (0.0032 mol) of sodium tungstate is added after 2 h of addition time.

At the end of the metered addition, the mixture is stirred at 80° C. for a further 3 h and then cooled to room temperature. Extraction with three 100 g portions of methylene chloride and concentration of the combined extracts affords 44.7 g of a pale beige solid comprising 85.4% of 1,4-dioxaspiro[4.5]decan-8-one (II-1), 7.8% of 1,4-cyclohexanedione (III) and 1.9% of 4-hydroxycyclohexanone (IV) (approx. 77% of theory).

### Example 2: Preparation of 1,4-dioxaspiro[4.5]decan-8-one

A mixture of 53.7 g (93%, 0.32 mol) of 1,4-dioxaspiro[4.5]decan-8-ol, 75 g of water, 1.3 g (0.016 mol) of sodium acetate and 2.0 g (0.006 mol) of sodium tungstate dihydrate is heated to 80° C. At this temperature, 65.7 g (0.97 mol) of 50% hydrogen peroxide is metered in over a period of 5 h. During this time, the pH of the reaction mixture is on reaching pH 6.0 always raised to pH 7.0 by adding 50% sodium hydroxide solution. In addition, a further 1.0 g (0.003 mol) of sodium tungstate is added after 2 h of addition time.

At the end of the metered addition, the mixture is stirred at 80° C. for a further 3 h and then cooled to room temperature. Extraction with three 100 g portions of methylene chloride and concentration of the combined extracts affords 46.2 g of a beige-brown solid comprising 82.0% of 1,4-dioxaspiro[4.5]decan-8-one (II-1), 3.7% of 1,4-cyclohexanedione (III) and 2.2% of 4-hydroxycyclohexanone (IV) (approx. 77% of theory).

### Example 3: Preparation of 1,4-dioxaspiro[4.5]decan-8-one

A mixture of 53.7 g (93%, 0.32 mol) of 1,4-dioxaspiro[4.5]decan-8-ol, 75 g of water, 1.3 g (0.016 mol) of sodium acetate and 2.0 g (0.006 mol) of sodium tungstate dihydrate is heated to 80° C. At this temperature, 65.7 g (0.97 mol) of 50% hydrogen peroxide is metered in over a period of 5 h. During this time, the pH of the reaction mixture is on reaching pH 6.5 always raised to pH 7.5 by adding 50% sodium hydroxide solution. In addition, a further 1.0 g (0.003 mol) of sodium tungstate is added after 2 h of addition time.

At the end of the metered addition, the mixture is stirred at 80° C. for a further 3 h and then cooled to room temperature. Extraction with three 100 g portions of methylene chloride and concentration of the combined extracts affords 44.4 g of a beige-brown solid comprising 82.8% of 1,4-dioxaspiro[4.5]decan-8-one (II-1), 1.7% of 1,4-cyclohexanedione (III) and 1.7% of 4-hydroxycyclohexanone (IV) (approx. 75% of theory).

### Example 4: Preparation of 1,4-dioxaspiro[4.5]decan-8-one

A mixture of 53.7 g (93%, 0.32 mol) of 1,4-dioxaspiro[4.5]decan-8-ol, 75 g of water, 1.3 g (0.016 mol) of sodium acetate and 2.0 g (0.006 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 45.8 g of a beige-brown solid comprising 86.7% of 1,4-dioxaspiro[4.5]decan-8-one (II-1), 3.3% of 1,4-cyclohexanedione (III) and 1.7% of 4-hydroxycyclohexanone (IV) (approx. 80% of theory).

### Example 5: Preparation of 1,4-dioxaspiro[4.5]decan-8-one

A mixture of 53.7 g (93%, 0.32 mol) of 1,4-dioxaspiro[4.5]decan-8-ol, 75 g of water, 1.3 g (0.016 mol) of sodium acetate and 1.0 g (0.003 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 42.9 g of a colourless solid comprising 72.8% of 1,4-dioxaspiro[4.5]decan-8-one (II-1), 2.0% of 1,4-cyclohexanedione (III) and 2.9% of 4-hydroxycyclohexanone (IV) (approx. 63% of theory).

### Example 6: Preparation of 1,4-dioxaspiro[4.5]decan-8-one

A mixture of 53.7 g (93%, 0.32 mol) of 1,4-dioxaspiro[4.5]decan-8-ol, 75 g of water, 1.3 g (0.016 mol) of sodium acetate and 4.0 g (0.012 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 44.9 g of a yellowish beige solid comprising 89.7% of 1,4-dioxaspiro[4.5]decan-8-one (II-1), 5.1% of 1,4-cyclohexanedione (III) and 1.1% of 4-hydroxycyclohexanone (IV) (approx. 82% of theory).

### Example 7: Preparation of 1,4-dioxaspiro[4.5]decan-8-one

A mixture of 53.7 g (93%, 0.32 mol) of 1,4-dioxaspiro[4.5]decan-8-ol, 75 g of water, 1.3 g (0.016 mol) of sodium acetate and 2.0 g (0.006 mol) of sodium tungstate dihydrate is heated to 75° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 45.1 g of a beige solid comprising 78.5% of 1,4-dioxaspiro[4.5]decan-8-one (II-1), 3.5% of 1,4-cyclohexanedione (III) and 2.4% of 4-hydroxycyclohexanone (IV) (approx. 72% of theory).

### Example 8: Preparation of 1,4-dioxaspiro[4.5]decan-8-one

A mixture of 53.7 g (93%, 0.32 mol) of 1,4-dioxaspiro[4.5]decan-8-ol, 75 g of water, 1.3 g (0.016 mol) of sodium acetate and 2.0 g (0.006 mol) of sodium tungstate dihydrate is heated to 85° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 44.2 g of a colourless solid comprising 86.6% of 1,4-dioxaspiro[4.5]decan-8-one (II-1), 7.4% of 1,4-cyclohexanedione (III) and 1.2% of 4-hydroxycyclohexanone (IV) (approx. 78% of theory).

### Example 9: Preparation of 1,4-dioxaspiro[4.5]decan-8-one

The pH of a mixture of 53.7 g (93%, 0.32 mol) of 1,4-dioxaspiro[4.5]decan-8-ol, 75 g of water, 1.3 g (0.016 mol) of sodium acetate and 2.0 g (0.006 mol) of sodium tungstate dihydrate is lowered to pH 7.0 by adding 99-100% acetic acid. After heating to 80° C., 65.7 g (0.97 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 41.8 g of a beige solid comprising 83.6% of 1,4-dioxaspiro[4.5]decan-8-one (II-1), 7.9% of 1,4-cyclohexanedione (III) and 2.3% of 4-hydroxycyclohexanone (IV) (approx. 71% of theory).

### Example 10: Preparation of 1,4-dioxaspiro[4.5]decan-8-one

The pH of a mixture of 44.0 g (90.9%, 0.25 mol) of 1,4-dioxaspiro[4.5]decan-8-ol, 60 g of water, 4.0 g (0.048 mol) of sodium acetate and 1.7 g (0.006 mol) of sodium tungstate dihydrate is lowered to pH 7.2-7.5 by adding 99-100% acetic acid. After heating to 80° C., 50 g (0.74 mol) of 50% hydrogen peroxide is metered in over a period of 4 h.

At the end of the metered addition, the mixture is stirred at 80° C. for a further 1 h and then cooled to room temperature. Extraction with three 30 g portions of methylene chloride and concentration of the combined extracts affords 32.5 g of a pale beige solid comprising 92.4% of 1,4-dioxaspiro[4.5]decan-8-one (II-1), 2.5% of 1,4-cyclohexanedione (III) and 0.8% of 4-hydroxycyclohexanone (IV) (approx. 76% of theory).

### Example 11: Preparation of 1,4-dioxaspiro[4.5]decan-8-one

A mixture of 53.7 g (93%, 0.32 mol) of 1,4-dioxaspiro[4.5]decan-8-ol, 75 g of water, 1.2 g (0.016 mol) of ammonium acetate and 2.0 g (0.006 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 43.4 g of a beige-brown solid comprising 83.6% of 1,4-dioxaspiro[4.5]decan-8-one (II-1), 7.5% of 1,4-cyclohexanedione (III) and 2.2% of 4-hydroxycyclohexanone (IV) (approx. 74% of theory).

### Example 12: Preparation of 1,4-dioxaspiro[4.5]decan-8-one

A mixture of 53.7 g (93%, 0.32 mol) of 1,4-dioxaspiro[4.5]decan-8-ol, 75 g of water, 1.2 g (0.016 mol) of ammonium acetate and 2.0 g (0.006 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 25% aqueous ammonia.

At the end of the metered addition, the mixture is stirred at 80° C. for a further 3 h and then cooled to room temperature. Extraction with three 100 g portions of methylene chloride and concentration of the combined extracts affords 40.8 g of a brown solid comprising 83.6% of 1,4-dioxaspiro[4.5]decan-8-one (II-1), 5.9% of 1,4-cyclohexanedione (III) and 1.7% of 4-hydroxycyclohexanone (IV) (approx. 69% of theory).

### Example 13: Preparation of 1,4-dioxaspiro[4.5]decan-8-one

A mixture of 53.7 g (93%, 0.32 mol) of 1,4-dioxaspiro[4.5]decan-8-ol, 75 g of water, 1.3 g (0.016 mol) of sodium acetate and 1.3 g (0.006 mol) of sodium molybdate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. An in-process control affords a mixture of 30.1% of 1,4-dioxaspiro[4.5]decan-8-one (II-1), 61.3% of 1,4-dioxaspiro[4.5]decan-8-ol (I-1), 0.4% of 1,4-cyclohexanedione (III) and 7.0% of 4-hydroxycyclohexanone (IV). The batch was not worked up.

### Example 14: Preparation of 1,4-dioxaspiro[4.5]decan-8-one

A mixture of 53.7 g (93%, 0.32 mol) of 1,4-dioxaspiro[4.5]decan-8-ol, 75 g of water, 1.0 g (0.016 mol) of lithium acetate and 2.0 g (0.006 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 44.9 g of an almost colourless solid comprising 82.5% of 1,4-dioxaspiro[4.5]decan-8-one (II-1), 1.6% of 1,4-cyclohexanedione (III) and 1.9% of 4-hydroxycyclohexanone (IV) (approx. 75% of theory).

### Example 15: Preparation of 1,4-dioxaspiro[4.5]decan-8-one

A mixture of 53.7 g (93%, 0.32 mol) of 1,4-dioxaspiro[4.5]decan-8-ol, 75 g of water, 1.0 g (0.016 mol) of lithium acetate and 2.0 g (0.006 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 10% lithium hydroxide solution.

At the end of the metered addition, the mixture is stirred at 80° C. for a further 3 h and then cooled to room temperature. Extraction with three 100 g portions of methylene chloride and concentration of the combined extracts affords 40.2 g of a colourless solid comprising 86.9% of 1,4-dioxaspiro[4.5]decan-8-one (II-1), 4.3% of 1,4-cyclohexanedione (III) and 1.4% of 4-hydroxycyclohexanone (IV) (approx. 71% of theory).

### Example 16: Preparation of 1,4-dioxaspiro[4.5]decan-8-one

A mixture of 53.7 g (93%, 0.32 mol) of 1,4-dioxaspiro[4.5]decan-8-ol, 75 g of water, 1.0 g (0.016 mol) of potassium acetate and 2.0 g (0.006 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% potassium hydroxide solution.

At the end of the metered addition, the mixture is stirred at 80° C. for a further 3 h and then cooled to room temperature. Extraction with three 100 g portions of methylene chloride and concentration of the combined extracts affords 44.6 g of an almost colourless solid comprising 84.9% of 1,4-dioxaspiro[4.5]decan-8-one (II-1), 4.4% of 1,4-cyclohexanedione (III) and 1.9% of 4-hydroxycyclohexanone (IV) (approx. 77% of theory).

### Example 17: Preparation of 1,4-dioxaspiro[4.5]decan-8-one

A mixture of 53.7 g (93%, 0.32 mol) of 1,4-dioxaspiro[4.5]decan-8-ol, 75 g of water, 1.0 g (0.016 mol) of sodium propionate and 2.0 g (0.006 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 44.9 g of a yellow-beige solid comprising 83.8% of 1,4-dioxaspiro[4.5]decan-8-one (II-1), 4.4% of 1,4-cyclohexanedione (III) and 2.0% of 4-hydroxycyclohexanone (IV) (approx. 76% of theory).

### Example 18: Preparation of 1,4-dioxaspiro[4.5]decan-8-one

A mixture of 53.7 g (93%, 0.32 mol) of 1,4-dioxaspiro[4.5]decan-8-ol, 75 g of water, 1.0 g (0.016 mol) of sodium butyrate and 2.0 g (0.006 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 46.0 g of a colourless solid comprising 82.1% of 1,4-dioxaspiro[4.5]decan-8-one (II-1), 4.1% of 1,4-cyclohexanedione (III) and 2.1% of 4-hydroxycyclohexanone (IV) (approx. 77% of theory).

### Example 19: Preparation of 1,4-dioxaspiro[4.5]decan-8-one

To a mixture of 75 g of water and 2.0 g (0.006 mol) of sodium tungstate dihydrate is added at room temperature 1.4 g (0.016 mol) of isobutyric acid and then 2.0 g (0.016 mol) of 32% sodium hydroxide. 53.7 g (93%, 0.32 mol) of 1,4-dioxaspiro[4.5]decan-8-ol is added and the mixture is then heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 46.3 g of a yellow-beige solid comprising 83.4% of 1,4-dioxaspiro[4.5]decan-8-one (II-1), 4.2% of 1,4-cyclohexanedione (III) and 2.1% of 4-hydroxycyclohexanone (IV) (approx. 78% of theory).

### Example 20: Preparation of 1,4-dioxaspiro[4.5]decan-8-one

A mixture of 43.1 g (92.8%, 0.25 mol) of 1,4-dioxaspiro[4.5]decan-8-ol, 60 g of water, 1.0 g (0.012 mol) of sodium acetate and 1.7 g (0.005 mol) of sodium tungstate dihydrate is heated to 80° C. At this temperature, 38 g (0.56 mol) of 50% hydrogen peroxide s metered in over a period of 3 h, followed by a further 22 g (0.32 mol) of 50% hydrogen peroxide over a period of 2 h. During this time, the pH of the reaction mixture is on reaching pH 5.5 each time always raised to pH 6.5 by adding 50% sodium hydroxide solution.

At the end of the metered addition, the mixture is stirred at 80° C. for a further 2 h and then cooled to room temperature. Extraction with three 30 g portions of methylene chloride and concentration of the combined extracts affords 34.9 g of a pale beige solid comprising 92.9% of 1,4-dioxaspiro[4.5]decan-8-one (II-1), 3.5% of 1,4-cyclohexanedione (III) and 0.9% of 4-hydroxycyclohexanone (IV) (approx. 82% of theory).

### Example 21: Preparation of 1,4-dioxaspiro[4.5]decan-8-one

A mixture of 53.7 g (93%, 0.32 mol) of 1,4-dioxaspiro[4.5]decan-8-ol, 75 g of water, 1.3 g (0.016 mol) of sodium acetate and 2.0 g (0.006 mol) of sodium tungstate dihydrate is heated to 80° C. At this temperature, 72 g (1.06 mol) of 50% hydrogen peroxide is metered in over a period of 3 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 45.1 g of a beige-brown solid comprising 76.9% of 1,4-dioxaspiro[4.5]decan-8-one (II-1), 16.5% of 1,4-dioxaspiro[4.5]decan-8-ol (I-1), 2.7% of 1,4-cyclohexanedione (III) and 2.6% of 4-hydroxycyclohexanone (IV) (approx. 70% of theory).

### Example 22: Preparation of 1,4-dioxaspiro[4.5]decan-8-one

A mixture of 53.7 g (93%, 0.32 mol) of 1,4-dioxaspiro[4.5]decan-8-ol, 75 g of water, 5.2 g (0.06 mol) of sodium acetate and 2.0 g (0.006 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 50.0 g of a beige solid comprising 76.0% of 1,4-dioxaspiro[4.5]decan-8-one (II-1), 12.9% of 1,4-dioxaspiro[4.5]decan-8-ol (I-1), 0.2% of 1,4-cyclohexanedione (III) and 2.6% of 4-hydroxycyclohexanone (IV) (approx. 77% of theory).

### Example 23: Preparation of 1,4-dioxaspiro[4.5]decan-8-one

A mixture of 53.7 g (93%, 0.32 mol) of 1,4-dioxaspiro[4.5]decan-8-ol, 75 g of water, 13.0 g (0.16 mol) of sodium acetate and 2.0 g (0.006 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 52.8 g of a beige-brown solid comprising 62.9% of 1,4-dioxaspiro[4.5]decan-8-one (II-1), 27.3% of 1,4-dioxaspiro[4.5]decan-8-ol (I-1), 0.1% of 1,4-cyclohexanedione (III) and 3.0% of 4-hydroxycyclohexanone (IV) (approx. 67% of theory).

### Example 24: Preparation of 1,4-dioxaspiro[4.5]decan-8-one

A mixture of 53.7 g (93%, 0.32 mol) of 1,4-dioxaspiro[4.5]decan-8-ol, 75 g of water, 1.3 g (0.016 mol) of sodium acetate and 10.0 g (0.030 mol) of sodium tungstate dihydrate is heated to 80° C. At this temperature, 72 g (1.06 mol) of 50% hydrogen peroxide is metered in over a period of 5 h. During this time, the pH of the reaction mixture is on reaching pH 5.5 always raised to pH 6.5 by adding 50% sodium hydroxide solution.

At the end of the metered addition, the mixture is stirred at 80° C. for a further 3 h and then cooled to room temperature. Extraction with three 100 g portions of methylene chloride and concentration of the combined extracts affords 43.4 g of a beige solid comprising 78.8% of 1,4-dioxaspiro[4.5]decan-8-one (II-1), 9.5% of 1,4-dioxaspiro[4.5]decan-8-ol (I-1), 0.0% of 1,4-cyclohexanedione (III) and 1.4% of 4-hydroxycyclohexanone (IV) (approx. 69% of theory).

### Comparative Example 1

### Preparation of 1,4-dioxaspiro[4.5]decan-8-one in analogous manner to Noyori et al.; Bull. Chem. Soc. Japan, 72(10), 1999, 2287-2306

A mixture of 53.7 g (93%, 0.32 mol) of 1,4-dioxaspiro[4.5]decan-8-ol, 75 g of water and 2.0 g (0.006 mol) of sodium tungstate dihydrate is heated to 80° C. At this temperature, 54 g (0.79 mol) of 50% hydrogen peroxide is metered in over a period of 6 h.

At the end of the metered addition, the mixture is stirred at 80° C. for a further 2 h, then cooled to room temperature and the pH adjusted to pH 6.0-6.5 with sodium hydrogen carbonate. Extraction with three 100 g portions of methylene chloride and concentration of the combined extracts affords 22.6 g of a colourless solid comprising 38.4% of 1,4-dioxaspiro[4.5]decan-8-one (II-1), 3.0% of 1,4-dioxaspiro[4.5]decan-8-ol (I-1), 30.3% of 1,4-cyclohexanedione (III) and 5.3% of 4-hydroxycyclohexanone (IV) (approx. 18% of theory).

### Comparative Example 2

### Preparation of 1,4-dioxaspiro[4.5]decan-8-one in analogous manner to Noyori et al.; Bull. Chem. Soc. Japan, 72(10), 1999, 2287-2306

A mixture of 53.7 g (93%, 0.32 mol) of 1,4-dioxaspiro[4.5]decan-8-ol, 75 g of water and 2.0 g (0.006 mol) of sodium tungstate dihydrate is heated to 80° C. At this temperature, 72 g (1.06 mol) of 50% hydrogen peroxide is metered in over a period of 6 h.

At the end of the metered addition, the mixture is stirred at 80° C. for a further 2 h, then cooled to room temperature and the pH adjusted to pH 6.0-6.5 with sodium hydrogen carbonate. Extraction with three 100 g portions of methylene chloride and concentration of the combined extracts affords 7.9 g of a colourless solid comprising 41.0% of 1,4-dioxaspiro[4.5]decan-8-one (II-1), 0.5% of 1,4-dioxaspiro[4.5]decan-8-ol (I-1), 50.9% of 1,4-cyclohexanedione (III) and 3.7% of 4-hydroxycyclohexanone (IV) (approx. 7% of theory).

### Comparative Example 3

### Preparation of 1,4-dioxaspiro[4.5]decan-8-one in analogous manner to Noyori et al.; Bull. Chem. Soc. Japan, 72(10), 1999, 2287-2306

A mixture of 53.7 g (93%, 0.32 mol) of 1,4-dioxaspiro[4.5]decan-8-ol, 75 g of water and 2.0 g (0.006 mol) of sodium tungstate dihydrate is heated to 80° C. At this temperature, 72 g (1.06 mol) of 50% hydrogen peroxide is metered in over a period of 3 h.

At the end of the metered addition, the mixture is stirred at 80° C. for a further 4 h, then cooled to room temperature and the pH adjusted to pH 6.0-6.5 with sodium hydrogen carbonate. Extraction with three 100 g portions of methylene chloride and concentration of the combined extracts affords 22.7 g of a colourless solid comprising 58.3% of 1,4-dioxaspiro[4.5]decan-8-one (II-1), 1.3% of 1,4-dioxaspiro[4.5]decan-8-ol (I-1), 25.5% of 1,4-cyclohexanedione (III) and 4.8% of 4-hydroxycyclohexanone (IV) (approx. 27% of theory).

1. A method for preparing compounds of the formula (II) 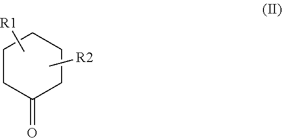
in which
R¹ and R² and the carbon atom to which they are attached represent a five- or six-membered ketal that is optionally singly or doubly substituted by methyl or ethyl,
characterized in that the compound of the formula (I) 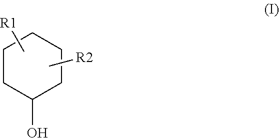
in which R¹ and R² are as defined above,
reacts with hydrogen peroxide in the presence of a solvent, in the presence of a catalyst, in the presence of a buffer and optionally in the presence of a base.

2. The method for preparing compounds of the formula (II) according to claim 1, in which
R¹ and R² and the carbon atom to which they are attached represent a five-membered ketal.

3. The method for preparing compounds of the formula (II) according to claim 1, in which the compound of the formula (I-1) is 1,4-dioxaspiro[4.5]-decan-8-ol and the compound of the formula (II-1) is 1,4-dioxaspiro[4.5]-decan-8-one.

4. The method according to claim 1, wherein sodium tungstate or sodium tungstate dihydrate is used as catalyst.

5. The method according to claim 1, wherein sodium acetate, ammonium acetate, lithium acetate, potassium acetate, sodium propionate, sodium butyrate or sodium isobutyrate is used as buffer.

6. The method according to claim 1, wherein sodium acetate is used as buffer.

7. The method according to claim 1, wherein aqueous ammonia, sodium hydroxide, potassium hydroxide or lithium hydroxide is used as base.

8. The method according to claim 1, wherein sodium hydroxide is used as base.

9. The method according to claim 1, wherein the pH of the reaction mixture is maintained between 5.5 and 7.5.

10. The method according to claim 1, wherein the pH of the reaction mixture is maintained between 5.5 and 6.5.

11. The method according to claim 1, wherein the oxidant is used in amounts of 1 to 5 mol per mole of compound of the formula (I).

12. The method according to claim 1, wherein the oxidant is used in amounts of 2 to 4 mol per mole of compound of the formula (I).

13. The method according to claim 1, wherein the oxidant is added over a period of 3 to 5 hours.

14. The method according to claim 1, wherein the buffer is used in amounts of 0.01 to 1 mol per mole of compound of the formula (I).

15. The method according to claim 1, wherein the catalyst is used in amounts of 0.01 to 0.2 mol per mole of compound of the formula (I).

16. The method according to claim 1, wherein water is used as solvent.

17. The method according to claim 1, in the presence of water as solvent, in the presence of sodium tungstate or sodium tungstate dihydrate as catalyst, in the presence of a buffer and optionally in the presence of a base, wherein the pH of the reaction mixture is maintained between 5.5 and 7.5.
